(19) United States
(12) Patent Application Publication
(10) Pub. No.: US 2020/0074884 A1
(43) Pub. Date: Mar. 5, 2020

(54) SYSTEMS AND METHODS FOR CREATING MEAL PLANS

(71) Applicant: NUTRISTYLE INC., Meridian, ID (US)

(72) Inventors: Scott Murdoch, Bend, OR (US); Todd Albro, Eagle, ID (US); Caleb Skinner, Beaverton, OR (US); Shannon Madsen, Livermore, CA (US); Lee Brillhart, Seattle, WA (US)

(21) Appl. No.: 16/557,983

(22) Filed: Aug. 30, 2019

Related U.S. Application Data
(60) Provisional application No. 62/725,334, filed on Aug. 31, 2018.

(51) Int. Cl. G09B 19/00; G16H 20/60

(52) U.S. Cl. CPC G09B 19/0092; G16H 20/60

(57) ABSTRACT

A method of operating a meal plan optimization algorithm involves retrieving at least one user profile from a profile database. A menu generation algorithm is configured with user preferences from the at least one user profile. A selector is configured to retrieve food items from at least one proximal food database for the menu generation algorithm from the user preferences. The menu generation algorithm may operate a meal target evaluator recursively, through operation of an iterator, to adjust and substitute meal components and then generate a meal day plan from the individual meals. A kcal target evaluator may be operated to adjust the portion size for the individual meals in the meal day plan. And a food menu for at least one day displayable through a user interface associated with the at least one user profile, from meal day plans may be generated through operation of a constructor.

### BACKGROUND

Consistent, defined period meal planning allows users to stick to their dietary constraints by limiting unplanned meals that may not fit their nutritional or caloric targets for the particular meal, day, week, and/or other defined period, or which they don't like or can't afford. While meal planning is useful, many individuals find the process of choosing meals and ingredients that fit within their dietary constraints and meet other objectives to be tedious and complex. This may be due to various reasons including finding meals that meet their food preferences, finding the ingredients for those meals, and adjusting portion sizes to meet the nutrient and caloric (kilocalorie or “kcal”) targets for the meal and/or day. Therefore, a need exists for improving the process of planning meals for a particular defined period.

### BRIEF DESCRIPTION OF THE SEVERAL VIEWS OF THE DRAWINGS

To easily identify the discussion of any particular element or act, the most significant digit or digits in a reference number refer to the figure number in which that element is first introduced.

FIG. 1 illustrates a system 100 in accordance with some embodiments.

FIG. 2 illustrates a method 200 in accordance with some embodiments.

FIG. 3 illustrates a system 300 in accordance with some embodiments.

FIG. 4 illustrates a system 400 in accordance with some embodiments.

FIG. 5 illustrates a system 500 in accordance with some embodiments.

FIG. 6 illustrates a system 600 in accordance with some embodiments.

FIG. 7 illustrates a system 700 in accordance with some embodiments.

FIG. 8 illustrates a system 800 in accordance with some embodiments.

FIG. 9 illustrates a system 900 in accordance with some embodiments.

FIG. 10 illustrates a system 1000 in accordance with some embodiments.

FIG. 11 illustrates a system 1100 in accordance with some embodiments.

FIG. 12 illustrates a system 1200 in accordance with some embodiments.

### DETAILED DESCRIPTION

“Food” refers to any substance consumed to provide nutritional support for an organism. For example, foods may be an assortment of consumable substances that include meats, grains, dairy products, fruits, mushrooms, vegetables, any plants, animals, insects, microbes, and any isolated or modified component of these. The foods may include condiments such as spices that may be added in combination to the aforementioned foods. Furthermore, foods may include beverages. Individual foods may be combined as components of a meal.

“Meal” refers to a single food component or combination of food components served individually or in combinations as a dish. A meal may include a dish of a variety of food components and spices accompanied by a beverage.

“Nutrient” refers to a substance used by an organism to survive, grow, and reproduce. The requirement for dietary nutrient intake applies to animals, plants, fungi, and protists. Nutrients can be incorporated into cells for metabolic purposes or excreted by cells to create non-cellular structures, such as hair, scales, feathers, or exoskeletons. Some nutrients can be metabolically converted to smaller molecules in the process of releasing energy, such as for carbohydrates, lipids, proteins, and fermentation products (ethanol or vinegar), leading to end-products of water and carbon dioxide. Nutrients include both macronutrients and micronutrients. Macronutrients provide energy and are chemical compounds that humans consume in the largest quantities and provide bulk energy are classified as carbohydrates, proteins, and fats. Water must be also consumed in large quantities. Micronutrients support metabolism and include dietary minerals and vitamins. Dietary minerals are generally trace elements, salts, or ions such as copper and iron. Some of these minerals are essential to human metabolism. Vitamins are organic compounds essential to the body. They usually act as coenzymes or cofactors for various proteins in the body. Nutrients also include bioactive compounds and nutraceuticals, which may be compounds found in foods, are not necessarily synthesized by the body, and are not directly involved in any fundamental functions of the body, yet can alter various metabolic functions within the body to impact health or disease. Some of these nutrients may include lipoic acid, ubiquinones (e.g., CoQ10, carotenoids, phenolic compounds, and the like). Other nutrients impact the functional characteristics of foods, which is defined by how the nutrients impact the consumer. For example, foods of this type include nutrients which impact the glycemic index/load which determines the impact of the food in causing increased blood glucose and/or insulin levels and acid/alkali forming which focuses on the impact on pH levels in the blood and cells, for example.

“Food Distributor” refers to any purveyor (e.g., grocery store, grocery delivery service, etc.,) that primarily offers ingredients to a user to utilize as the components of a meal, with the unit size of the ingredient being greater than the quantity required for an individual meal portion. A main difference between a food distributor and a restaurant/food service is in the quantity of the components usually exceeding the quantity required for a single meal.

A method of operating a meal plan optimization algorithm may involve retrieving at least one user profile from a profile database. A menu generation algorithm may be configured with user preferences from the at least one user profile. A selector may be configured to retrieve food items from at least one proximal food database for the menu generation algorithm from the user preferences. The menu generation algorithm configured by the user preferences may be operated to set preferred food items for meal components of individual meals from the food items in the at least one proximal food database through operation of a food preference discriminator, set a non-user specified food item for a meal component of the individual meals without a preferred food item and additional food items through operation of the food preference discriminator, and update a meal total for the individual meals with the non-user specified food item. The menu generation algorithm may operate a meal target evaluator recursively, through operation of an iterator, to adjust and substitute meal components. A meal day plan may be generated from the individual meals. A kcal target evaluator may be operated to adjust the portion size for the individual meals in the meal day plan configured by the user preferences. And a food menu for at least one day displayable through a user interface associated with the at least one user profile, from meal day plans may be generated through operation of a constructor.

In some configurations, the kcal target evaluator configured by the user preferences from each user profile may be operated to adjust the portion size of the individual meals in the meal day plan for each user profile

In some configurations, the user preferences include food dislikes, food likes, food allergies or restrictions, meal preferences, nutrient targets, weight or other personal health objectives, financial budget, preferred grocers or food distributors, preferred brands or private labels, and kcal target from the at least one user profile.

In some configurations, the method of operating a meal plan optimization algorithm involves retrieving at least one user profile from a profile database, configuring the menu generation algorithm with food dislikes, food likes, meal preferences, nutrient targets, financial budget, and kcal target from the at least one user profile, configuring a selector with location information from the at least one user profile to retrieve food items from at least one proximal food database for the menu generation algorithm, operating the menu generation algorithm to set preferred food items for meal components of individual meals from the food items in the at least one proximal food database through operation of a food preference discriminator configured by the meal preferences, setting a non-user specified food item for a meal component of the individual meals without a preferred food item and additional food items through operation of the food preference discriminator configured by the food likes and the food dislikes, operating a meal target evaluator, and generating a meal day plan from the individual meals. The method of operating a meal plan optimization algorithm further involves adjusting the portion size of the food items for the individual meals in the meal day plan through operation of the kcal target evaluator configured by the kcal target as specified by the user, and generating a 1-n-day food menu, displayable through a user interface associated with the at least one user profile, from meal day plans typically grouped together as menus through operation of a constructor. The method may further involve adjusting the portion size of the individual meals in the meal day plan for each user profile through operation of the kcal target evaluator configured by the kcal target from each user profile.

In the method of operating a meal plan optimization algorithm, the meal target evaluator iteratively operates, through operation of an iterator, to calculate nutrient gaps and nutrient target ratios, gap ratio score, scores and other mathematical relationships among the food items that comprise the individual meals adjust portion size of the food items within individual meals with the non-user specified food item in response to detecting the meal total score being outside of a meal target range based on the gap ratio score, substitute the non-user specified food item with different non-user specified food item in the individual meal with the closest meal total outside the meal target range, and substitute replace the food item with having the lowest nutrient value in the individual meal with the different non-user specified food item with the closest meal total outside the meal target range.

In some instances, these collections of food items may have their portion sizes adjusted; and, to achieve acceptable meal scores, can have non-user specified food items substituted for other non-user specified food items. This process of food item substitution is managed by any of a number of mathematically driven algorithms that are used to view the food item in question, determine the footprint of the food item to best fit the requirements of the meal and, finally, to manage the process of verifying that the food item substitution is a better fit for the meal.

In some configurations, the algorithm for generating a menu for a user through a user interface involves retrieving the menu preset for the user from the user's profile. Menu presets include 1-n parameters to assist in identifying a unique menu, including, without limitation, macronutrient (e.g., fat, protein, carbohydrate, alcohol) targets, food favorites (e.g., a specific brand of yogurt), food restrictions macronutrient settings for carbohydrate, fat, protein, and alcohol, and food dislikes. The kilocalorie (kcal) target for the user is also retrieved, as are 0-n micronutrient (e.g., amount of sodium or potassium) targets, as applicable. A check is performed to determine if additional user profiles will be involved in generating the menu. If additional profiles are included in the menu creation, the kcal target is increased to account for the additional profiles and a multiplier is determined to accommodate the additional individuals. After the adjustments have been made for the additional users, or if there are no additional users, foods are chosen. The profiles include, among other things, the user's likes and dislikes for certain foods, food restrictions, health objectives, financial budget, preferred food brands and/or private labels, and grocer or food distributor preferences. In some instances, the user may set a kcal target for individual meals, individual days of the defined period, or as the same kcal target for each day.

Once the user information has been collected, the menu generation process begins. The generated menu runs for 1-n meal cycles and may include standard meals (e.g., breakfast, lunch, dinner, and snacks (e.g., morning snack, afternoon snack, and evening snack)) that are ordered in the menu according to their chronological schedule. In some configurations, the meals may be ordered differently or based on user preference.

In some instances, previous menu configurations related to the user going over or under their predicted kcal target may be presented. When a new menu is generated, previous values are set to zero. For example, carry over of kcal, macronutrients, and micronutrients are reset to zero for a new menu. Food likes for individual meals or specific days are retrieved, as well as preset food likes, and preferred brands for individual meals or specific days. This preset allows users to specify specific foods at specific times in advance, for instance if the user exercises on certain days and needs a higher protein intake after their workout.

Components for the day/meal are retrieved at this point. The components may be a broad categorization of certain food items that go into a meal based on food preferences. Once the components are retrieved, the individual foods are retrieved for each component. The menu generation process then begins to perform an iterative process, going through each day and individual meals for that day, starting with the first day of meals until it reaches the final meal on the last day of the 1-n day defined period. The iterative or looped process involves randomly retrieving one food item per component, screening for various values included in the user profile, adding food likes to the meal, and updating the meal totals, and then determining the meal type. Depending on the meal type (e.g., traditional meals (e.g., breakfast, lunch, dinner) or snacks) the system may update values for the day or meal based on individual totals or modifications. This may occur 1-n times per meal component. If the meal type is a traditional meal (e.g., breakfast, lunch, dinner) gaps in the meals are calculated, such as the ratios of carbohydrates, fats, proteins, and alcohol. This process is then followed by a 1-n loop to adjust the portion size of the meals. If the meal type is a snack, the food item is added, and the snack totals are adjusted while adjusting the meal totals. Once the process of generating the menu is completed, the user profile is checked to determine the number (1-n) of users. If more than one user profile was included in the generation of the menu, the process creates a menu with appropriate adjustments to the portion size to reflect the kcal target of the user and then sets the previous menu's primary menu Boolean value to false. The process then creates a system food log day for each day of the menu. If more than one user profile is not involved in the menu generation, a menu is created with the appropriate adjustments to the portion sizes to reflect the kcal target for the user, and the previous menu's primary menu Boolean value is set to false removing the previous menu. The process then creates a food log day for each day of the menu.

The 1-n step loop adjustment for portion sizes of food components of a meal involves performing a gap ratio analysis from the food gaps created from the meal calculations following the addition of meal preferences and snacks. The gap ratio analysis checks the meal score. If the meal scores are correct, the system determines that the meal has been generated and then moves on to the next meal. If the meal score is not correct, and if the iteration completes without successfully hitting the meal target, the process checks the meal score. If the meal score is better than the existing value, the algorithm updates a score for the best scored meal combination with the current meal combinations score, and updates the best meal combination with the current meal combination. After completing 1-n number of iterations (e.g., 20), as optimized over time, the process determines if the best meal should be updated. If so, the process checks the meal totals again. Otherwise, the process moves on to a process for determining substitutions. The substitution process iterates through each food in the meal and does not process favorite food preferences. The process begins by removing food from the meal, followed by the first step a multistep iteration that runs in sets of 1-n until the process has iterated 1-n times. The process starts by looking for a food from the component collection that fits into the parameters within the set of iterations (loops). If a food item is found, a determination is made to see if the substitution works for the meal. If the substitution does not work, the food item is replaced with the original. If the substitution of the food item causes the meal to score better than the original, the food item is kept as a possible substitution, if the newly substituted food item makes the meal score worse, the process reverts back to the original food item. The process of reverting back to the original food item may also occur if no substitution is found.

Once the first step of the substitution is completed, and a substitution is not found, the food item is removed from the meal, the food sub category collection that the food item is part of is ordered based on the lowest value of needed macronutrients. The second step of the loop is then run where the food item is replaced by selecting a substitute food item from the ordered food sub category collection, the required calculations are then performed to determine the substitution. If the substitution does not work, a determination on the meal score is made. If the current meal score is better, the substituted food item is kept. If the current meal score is worse, the substituted food item is reverted back to the original food item and the process moves on to plan the next food item collection. If the substitution works, as determined by the meal score, the meal generation is completed, and the process moves on to the next meal.

A system running a meal plan nutrition optimization algorithm takes into account the preferences, dietary restrictions, health objectives, and financial budget set by the individual users and generates a single meal, series of meals, or 1-n day meal plan for the user. The optimization algorithm selects foods from local grocery store or food distributor inventories, considering a user's preferred food brands and private labels, and builds meals around them based on the totality of the user's preferences and the nutritional content of the selected food. The system provides users with a 1-n meal/day shopping or grocery. by taking those foods and listing the quantity of each food needed to build the list for the period defined by the user.

A user may set nutrient targets and preferences as well as set food items they want to include or exclude from their weekly menu of meals. The system then populates individual food items based on component categories to build meals that fit the user's target preferences within a narrow margin.

A user may set certain caloric and/or macronutrient and/or micronutrient targets for specific food items, meals and/or specific days and the system may then make adjustments to the individual meals to fit within the specified target within the meal or the day. The system may also obtain new targets for the day and then implement meals based on those targets. Additionally, the system may make adjustments to the meals based on a desired nutrient total/ratio that may cross reference the targets with the user's caloric target. The categorization of a meal (e.g., traditional meal (e.g., breakfast, lunch, and dinner) or a snack) may determine which targets (nutrient or kcal) are tracked and utilized in the adjustment of a daily target. For instance, the macro nutrient targets may not be utilized as a deciding factor to select a food item for a snack and may also not be utilized to influence the daily totals of the nutrient targets. However, the kcals in the snack may tracked and utilized to adjust portion sizes of the meals for that particular day.

In some configurations, as an example, the system may note that a user specified a brand or private label name soup on their profile, and then use that specified brand to find related foods items by that brand or private label to use in other meal options.

The system may also provide food variety to users by generating meals starting with a randomly selected food item. The randomly selected food item would be from a small pool of food items that meet the user's food preferences criteria, and then build a meal around that food item.

When setting up a food menu for a defined period, a user may specify a particular food item that they would like to use by entering the particular food item manually (e.g., typing or selecting a picture of the food item), by scanning the Universal Product Code (UPC) of that food item in their possession, by voice, or through other means.

By scanning the UPC, for example, the system may connect to a food item database associated with food items available at local grocery stores, local grocery store chains, or other nearby food distributors within the area, determined by the user's profile, or through location services, and determine whether the food item is in stock when generating the menu for the predetermined time period. This may be especially useful in determining whether to include seasonal food items in the user's weekly food menu. Regardless how the link to the grocer's or food distributor's inventory data is activated, food items will be identified not just by type, but by specific brand name (including private label or “store brands”) to capture the most accurate nutrition data for each food item. In addition, in certain embodiments, a local grocer or food distributor will be able to flag or otherwise identify products of which they have, for example, excess inventory, and have those items made extra visible to users through message or other process, including as a special promotion or coupon, or as a “special price” item when comparing meal and shopping list cost to the user specified financial budget. Such food promotions could be based, for example, on a specific branded or private label food item, a product category (e.g., yogurt), or a department (e.g., cheese) and the nature of the promotion(s) can be varied dynamically to assist grocers or food distributors in managing their inventory.

The system may also connect with the user's devices (e.g., wearable devices, mobile device, etc.) to determine the user's activity level. With the user's activity level, the system may be able to adjust the caloric target and/or portion size of the user's meals to more accurately reflect their dietary needs for that day.

The system may decide what foods to add to the user's menu based on an unassociated shopping list a user has created for a particular grocer. The system may view items within the list and plan meals based on the items on the list. The system may also provide users with suggestions on how to obtain the food items based on price or convenience. For example, the system may provide users with suggestions about the local grocers that may offer the food item at a lower price than other nearby grocers and/or provide the user with grocery delivery services that can deliver the food items to them.

The system may allow the user to go through their profile and set the preferences for the generated menu. A user may identify/update foods that they like for an already created menu. The system may then take those updated values and reconstruct the menu based on the user's updated preferences. The system may provide users with the ability to recalculate/reconfigure a specific meal vs the whole day by defining specific meals they would like to have ahead of time and/or by listing foods and/or food combinations that that they would like or not like to have for a particular meal. For instance, a user may specify that they would not like to have tuna for breakfast and the system may exclude tuna as a protein option for breakfast.

The food menu may also work in combination with a user food log or food tracker allowing a user to provide feedback on whether they have been eating the foods suggested by the food menu and also provide the food menu with the ability to adjust the meal options or portion size for the day in order to meet the user's nutrient targets. Users could enter data in the food log in multiple ways, including through standard keyboard entry, scanning the product code (e.g., UPC or QR), taking a picture of the food item/meal (with subsequent AI processing identifying the meal), voice, or other means.

The system may also provide users with health risk warnings letting the user know that they have consumed a specific food item or quantity that is not recommended for them. For instance, a user may set a beer as part of a snack or meal component, and the system may advise the user that beer may not be a good option for meeting their dietary targets.

In some configurations, the system selects foods based on their availability near the location of the user, then builds individual meals that meet the user's dietary constraints and food preferences. The system may also build food menus based on the number of individuals that a user is preparing food for. The system may define individual meals based on the number of users, especially if the users provide food preferences and dietary constraints. The system may go through the food preferences and dietary constraints of each user and generate a menu that would accommodate all their constraints. Additionally, the system may integrate with shopping list applications to determine food preferences and eating habits. For instance, the system may determine particular food likes and dislikes based on the foods or combination of foods that user has had on their shopping list, as well as the frequency that a user shops for those particular items.

The system may also integrate with a user's day planner or calendar and determine specific meals to offer to the user based on when the user is available. For instance, the system may determine that the user arrives at their home fairly late every day and may suggest certain meals that are easy and quick to prepare.

The system may also compare a user to other similar users based on, for example, their food preferences, dietary selections, shopping lists, consumption food logs, exercise type and schedule, food and/or brand favorites, food restrictions, etc. to suggest particular foods or avoid particular foods even though the user has not specified that they have a preference or lack thereof for that particular food item.

The system may also integrate with a food preference algorithm that identifies food pairings to suggest particular food combinations that a user may like to see in a meal.

In some configurations, the system may allow users to enter their prescription medication and supplement information and dosage schedule to help the system plan certain meals based on certain food drug interactions. For instance, grapefruit juice may inhibit the activity of certain digestive enzymes used for metabolizing orally administered medications. The system may identify the possible contraindications for the drugs listed by a user and exclude grapefruit and grapefruit juice containing foods from the user's meal plan. Additionally, the system may take into account the time when a user takes their medication to limit certain foods and/or quantities of macronutrients that may influence the efficacy of the medication if taken within a certain time period with each other. For instance, a user may take insulin and the system may identify meals to offer the user, at specific times, that may reduce the likelihood of their blood glucose level rising.

In some configurations, each daily menu may be broken down into a combination of meals with/without snack(s). A meal may be identified as group of foods that usually contains a beverage, primary food (aka a main entree), and a number of secondary foods that enhance the primary food. This collection of foods might have something sweet that would translate into a dessert. The food component make up of a meal varies as it may be different per ethnic group, region, place in the world, diet plan, etc. A snack may be identified and selected from a group of foods, typically one or two at most, that may be consumed between a meal.

In some configurations, the generation process treats the snack and meal process differently. The generation process treats the snack similarly to the meal process, with one exception: there may be usually only 1 Food Sub Category that the algorithm selects a single food item from (unlike a meal that contain several Food Sub Categories). So, the only thing that the generator process can do may be adjust the portion size of the snack. So, for a snack the process is: a while loop takes the collection of foods that comprise the snack and adjusts the portion size of each food. Each iteration of the loop retrieves a snack score, increases or decreases the portion size based on reviewing the values of the macronutrient values of the food. Once the adjustment may be made the snack may be scored. If the adjustment has caused the meal score to decrease then the adjustment may be kept and the process continues. The iterations continue until the snack score equals zero or the snack score increases as opposed to decreasing. These upper and lower targets can be adjusted based on the tuning of the generator.

In some configurations, the meal generation process may be a 1-n loop. The process may start with a collection of food components that comprise the meal. These food components may all have their portion sizes set to a typical portion amount. The first step of the process may be to determine a number of different mathematical ways of viewing the food components in relation to each other as well as in relation to the caloric target that may be being pursued. The following values may be calculated:
Carbohydrate gap=Abs(meal.CarbohydrateTarget)−meal. CarbohydrateActuals)
Fat gap=Abs(meal.FatTarget)−meal.FatActuals)
Protein gap=Abs(meal.ProteinTarget)−meal.ProteinActuals)
Ratio of the protein value to the value the meal target
Ratio of the fat value to the value of the meal target
Ration of the carbohydrate value to the value of the meal target
Carbohydrate gap percent
Fat gap percent
Protein gap percent
The ratio of carbohydrates to fat and protein
The ratio of fat to carbohydrates and protein
The ratio of protein to carbohydrates and fat
The carbohydrate variance=Abs(carbohydrateRatio−mealCarbohydarateRatio)
The fat variance=Abs(fatRatio−mealFatRatio)
The protein variance=Abs(proteinRatio−proteinRatio)

In some configurations, a ratio may be defined as the sum of the carb, fat and protein values divided by the specific nutrient(carbohydrate, fat, protein).

Once these values have been gathered the next step in the process may be to look at the collection of food components to find the appropriate food component to adjust relative to the target that may be trying to be achieved. This process iterates through the food component collection until either a successful meal has been generated or the 1-n iteration count may be breached.

If the adjustment of the portion size does not result in the successful generation of a meal the next step may be the process may be food substitution. This food substitution logic looks at the needed ratios and then sorts the available foods by them. The process increases the size of the circle around the food ratios until a match may be found. At this point a meal score may be taken, the food may be substituted in, the portion size adjusted and the meal may be again scored. If the substitution process results in a lower score the substitution may be considered a success and may be added as part of the meal. If the meal may be now inside a value range it may be considered generated successfully. If not the original food may be swapped back in and the process starts on the next food in the food component collection.

If a food substitution may be not found with this primary substitution process a secondary one in engaged. This process seeks to find a food for substitution based off of looking for the greatest value of carbohydrate, fat, protein and then sorts of the collection of available food substitutes in the appropriate order. This process returns a food. The process of scoring the meal, adjusting the portion sizes for the foods in the food component collection, and then scoring the resulting meal. If the score of the meal may be smaller the substitution may be considered a success. If the meal score falls into a range the meal generation may be considered a success.

In some configurations, the meal generation processes may carry over differences in the carbohydrate, fat, protein targets into the subsequent meal. This process smooths the meals throughout the day and assist with trying to hit specific targets with crude adjustment buckets. This carry over process does not calculate between days. It only moves between meal of the same day.

One of skill in the art will realize that the methods and apparatuses of this disclosure describe prescribed functionality associated with a specific, structured graphical interface. Specifically, the methods and apparatuses, inter alia, are directed to a system and method for operating a meal plan optimization algorithm that reduces the number of human interactions required to generate a typical weekly menu by automatically creating meals for the user that meet their nutritional and caloric targets. One of skill in the art will realize that these methods are significantly more than abstract data collection and manipulation.

Further, the methods provide a technological solution to a technological problem, and do not merely state the outcome or results of the solution. As an example, the system for operating a meal plan optimization algorithm reduces the number of human interactions required to generate a typical weekly menu by automatically creating meals for the user that meet their nutritional and caloric targets. This is a particular technological solution producing a technological and tangible result. The methods are directed to a specific technique that improves the relevant technology and are not merely a result or effect.

Additionally, the methods produce the useful, concrete, and tangible result of the generation and display of a weekly food menu, thereby identifying each change as associated with its antecedent rule set.

Further, the methods are directed to a specifically-structured graphical user interface, where the structure is coupled to specific functionality. More specifically, the methods disclose a specific set of information to the user, rather than using conventional user interface methods to display a generic index on a computer.

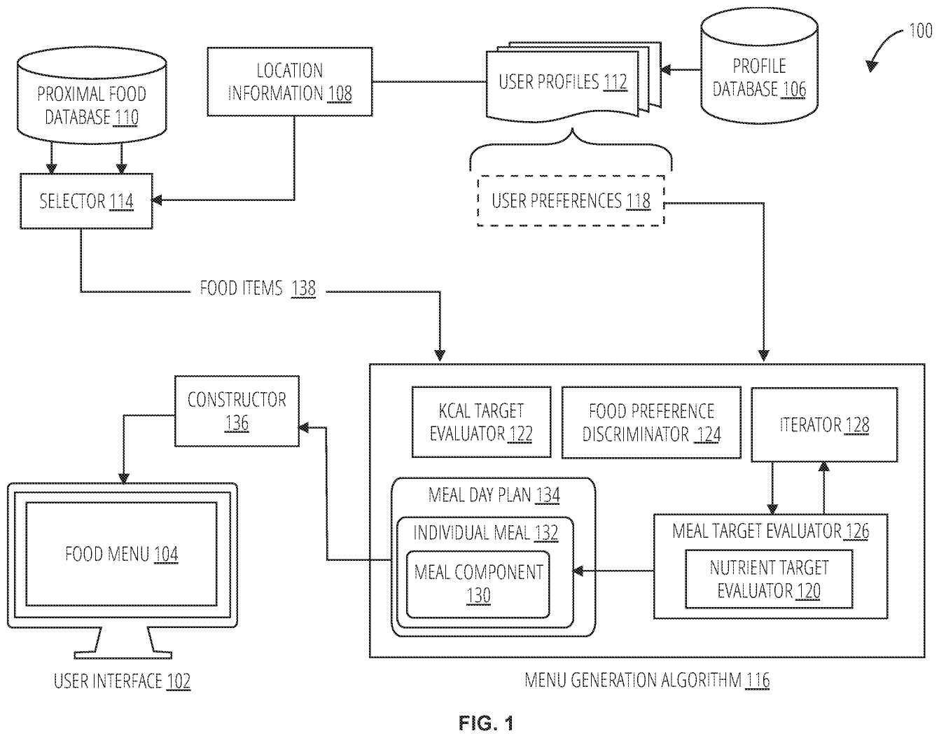

Referencing FIG. 1, a system 100 for operating a meal plan optimization algorithm includes a user interface 102, a profile database 106, a selector 114, a menu generation algorithm 116, and constructor 136. The profile database 106 comprises at least one user profile 112 each comprising location information 108 and user preferences 118. The location information 108 configures the selector 114, identifying at least one proximal food database to select food items 138 to generate the food menu for at least one day 104. The menu generation algorithm 116 receives the user preferences 118 from the at least one user profiles 112 and configures a kcal target evaluator 122, a food preference discriminator 124, and a meal target evaluator 126. The food preference discriminator 124 is configured to identify the food items 138 to use as meal component 130 for an individual meal 132. The menu generation algorithm 116 sets preferred food items for an individual meal 132. The meal target evaluator 126 utilizes the user preferences 118 to configure the nutrient target evaluator 120 to identify food item substitutions and portion size adjustments that best fit individual meals. With individual meals identified for a meal day plan 134, the menu generation algorithm 116 communicates the meal day plans to the constructor 136 which modifies the kcal target evaluator 122 to generate a food menu for at least one day 104 to be displayed through the user interface 102.

In some configurations, the user profile comprises user preferences comprising food preferences (i.e., likes/dislikes), food restrictions (e.g., gluten free), health objectives (e.g., lose weight), budget, preferred brands and/or private labels, and preferred grocers and/or food distributors.

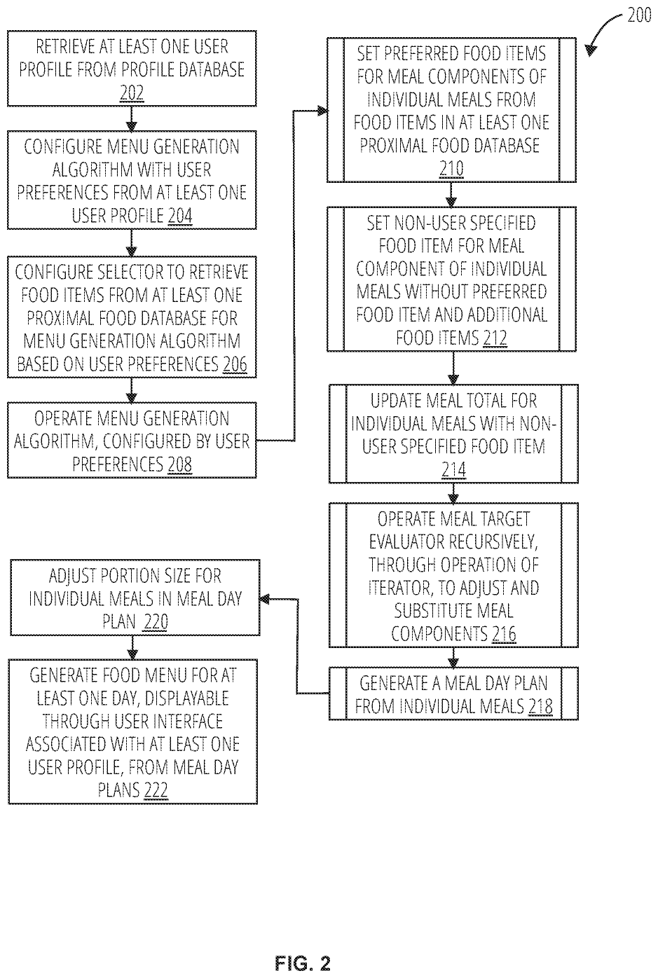

The system 100 may be operated in accordance with the processes described in FIG. 2.

Referencing FIG. 2, a method 200 for operating a meal plan nutrition optimization algorithm involves retrieving at least one user profile from a profile database (block 202). In block 204, the method 200 configures a menu generation algorithm with user preferences from the at least one user profile. In block 206, the method 200 configures a selector to retrieve food items from at least one proximal food database for the menu generation algorithm, from the user preferences. In block 208, the method 200 operates the menu generation algorithm, configured by the user preferences. In subroutine block 210, the menu generation algorithm sets preferred food items for meal components of individual meals from the food items in the at least one proximal food database through operation of a food preference discriminator. In subroutine block 212, the menu generation algorithm sets a non-user specified food item for a meal component of the individual meals without a preferred food item and additional food items through operation of the food preference discriminator. In subroutine block 214, the menu generation algorithm updates a meal total for the individual meals with the non-user specified food item. In subroutine block 216, the menu generation algorithm operates a meal target evaluator recursively, through operation of an iterator, to adjust and substitute meal components. In subroutine block 218, the menu generation algorithm generates a meal day plan from the individual meals. In block 220, the method 200 adjusts the portion size for the individual meals in the meal day plan through operation of the kcal target evaluator, configured by the user preferences. In block 222, the method 200 generates a food menu for at least one day, displayable through a user interface associated with the at least one user profile, from meal day plans through operation of a constructor.

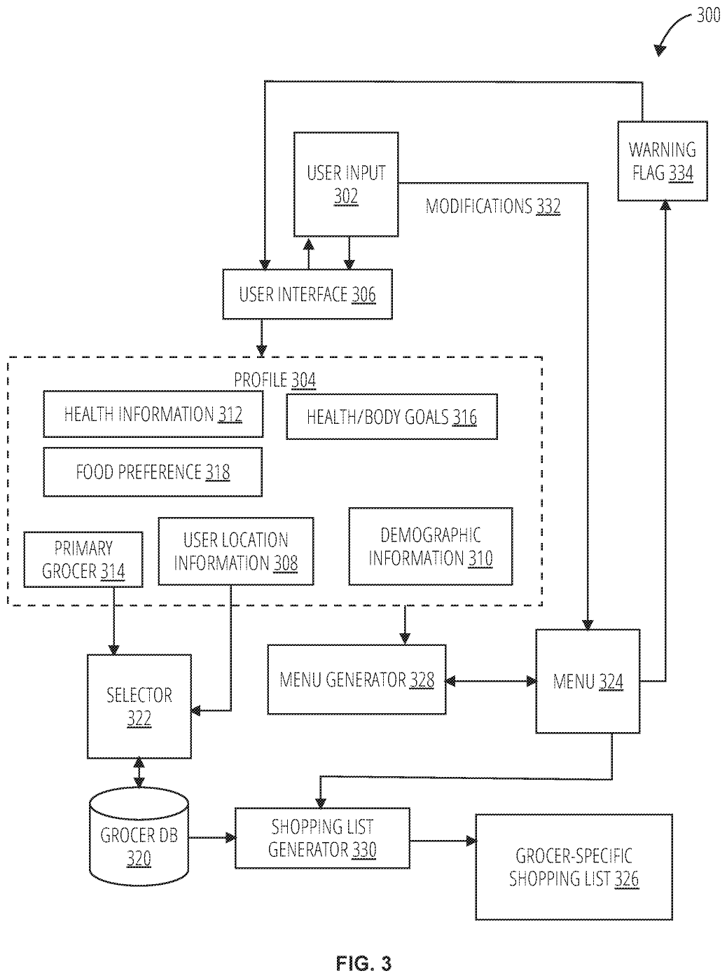

FIG. 3 illustrates an example system 300 that comprises a profile 304 that includes health information 312, food preference 318, health/body goals 316, primary grocer 314, user location information 308, and demographic information 310. The profile 304 is initially configured through a user interface 306 receiving user input 302. A selector 322 is configured utilizing the primary grocer 314 and user location information 308 to select a local grocer database 320. The menu generation algorithm 328 is configured utilizing the profile 304 to generate a menu 324. The local grocer database 320 and the menu 324 are utilized by a shopping list generator 330 to generate a grocer-specific shopping list 326. A warning flag 334 may be detected in the menu 324 that may be presented to the user through a user interface 306. The user may address the warning flag 334 in the menu 324 by entering modifications 332 to the menu 324. In some configurations, the warning flag 334 may be activated variously, including an immediate warning if a user enters a food item that is inconsistent with a user's food restrictions or is contraindicated based on nutritional supplements or medications taken by the user, or after a meal plan has been generated, to indicate, for example, that a meal exceeds the kcal target. The warning can be communicated in various ways, including in one embodiment a red, yellow, green system that indicates in a readily understandable way, whether a meal or food item is potentially dangerous (red), worthy of more consideration by the user (yellow), or “all clear” (green).

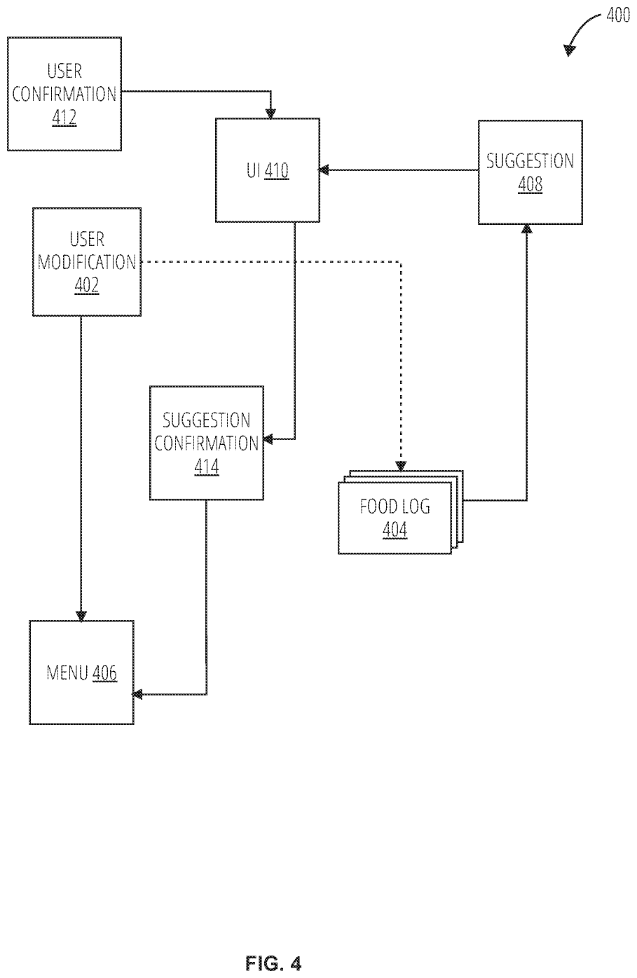

FIG. 4 illustrates an example system 400 that shows the interactions between a UI 410 that receives a suggestion 408 and a user confirmation 412 to generate a suggestion confirmation 414 regarding food items within a menu 406. The user modification 402 may change food items within the menu 406 which may also be utilized to populate a food log 404. The food log 404 may allow the system 400 to provide an improved suggestion 408 to the user through UI 410.

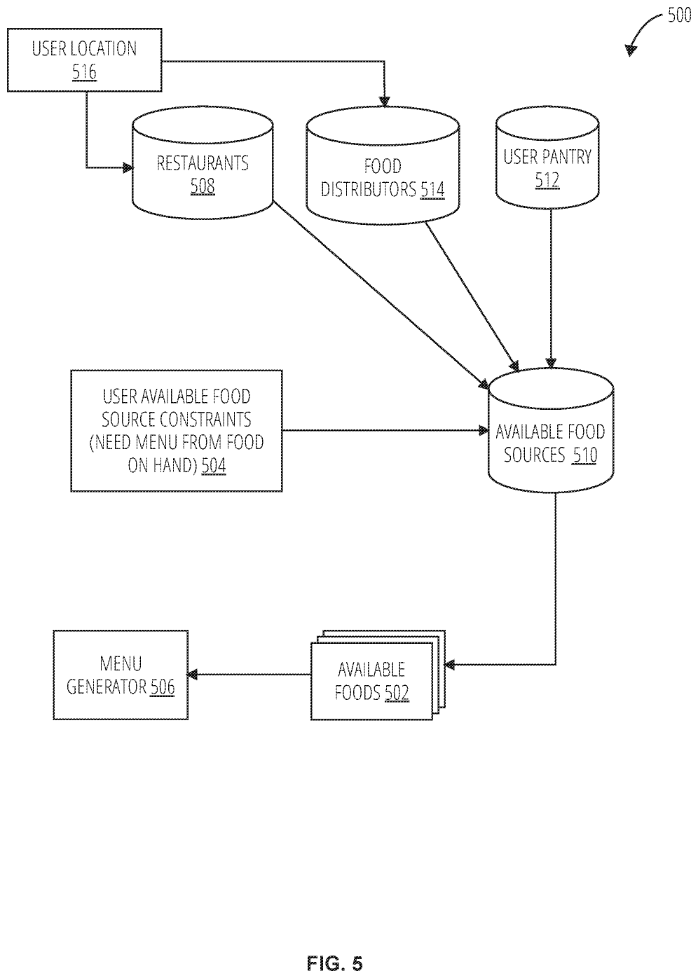

FIG. 5 illustrates an example system 500 that utilizes user location 516 to determine the restaurants 508 and the food distributors 514 nearby the user as available food sources 510 for generating meals. The system 500 may also utilize the contents within the user pantry 512 as an available food sources 510. In some instances, user available food source constraints (need menu from food on hand) 504 may require a user to modify their meals on their menus based on the available food sources 510 dependent on the user location 516. The available food sources 510 may be identified as available foods 502 from which the menu generator 506 may select to generate or modify the meal plan menu.

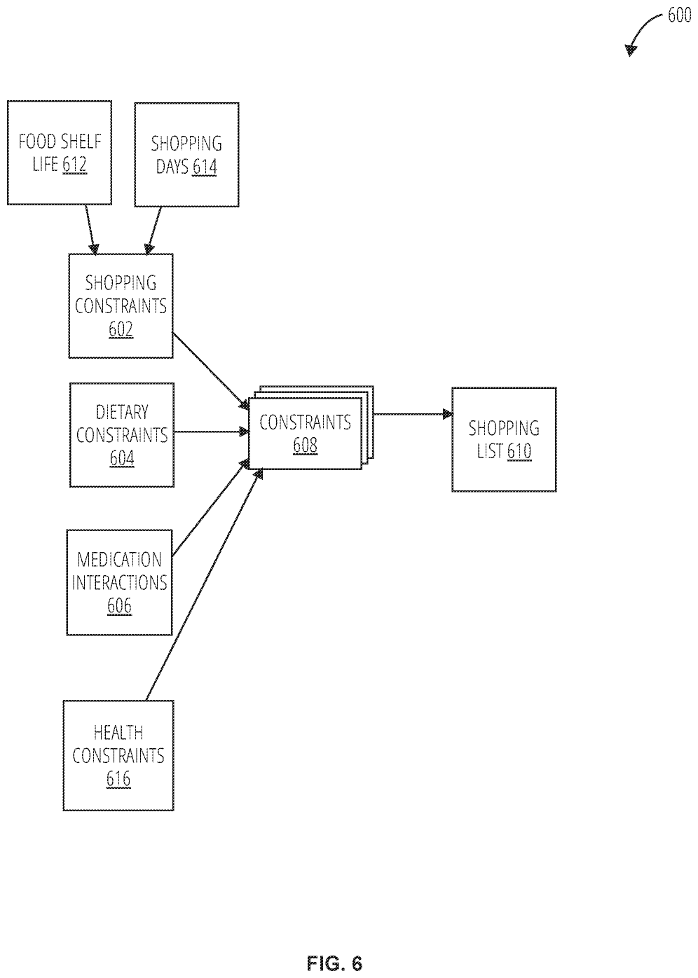

FIG. 6 illustrates an example system 600 that identifies constraints 608 that may be utilized in the generation of a shopping list 610. The constraints 608 includes shopping constraints 602 which may also include the food shelf life 612 and shopping days 614 available to the user, dietary constraints 604, medication interactions 606, and health constraints 616. In some configurations, the constraints 608 may include other limitations chosen by the user and/or imposed on the user.

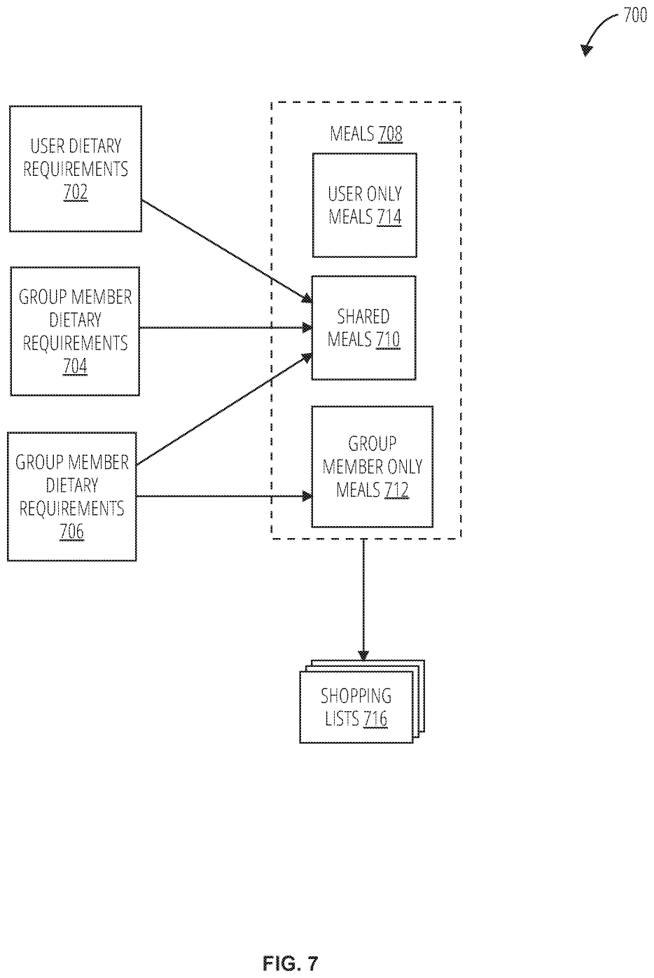

FIG. 7 illustrates an example system 700 showing the influencers for different categories of meals based on shared profiles. The meals 708 include user only meals 714, shared meals 710, and group member only meals 712. Group member only meals 712 may be any meal another member of the group eats that does not include other members of the group. When determining meals for multiple individuals in a group, the user dietary requirements 702 influence the shared meals 710, which may also be influenced by the group member dietary requirements 704 and the group member dietary requirements 706 of the other members of a family. For the other members of the group, their group member dietary requirements 706 may also affect group member only meals 712 that only the other member(s) eat. The meals 708 from all group members are then utilized to generate a shopping lists 716.

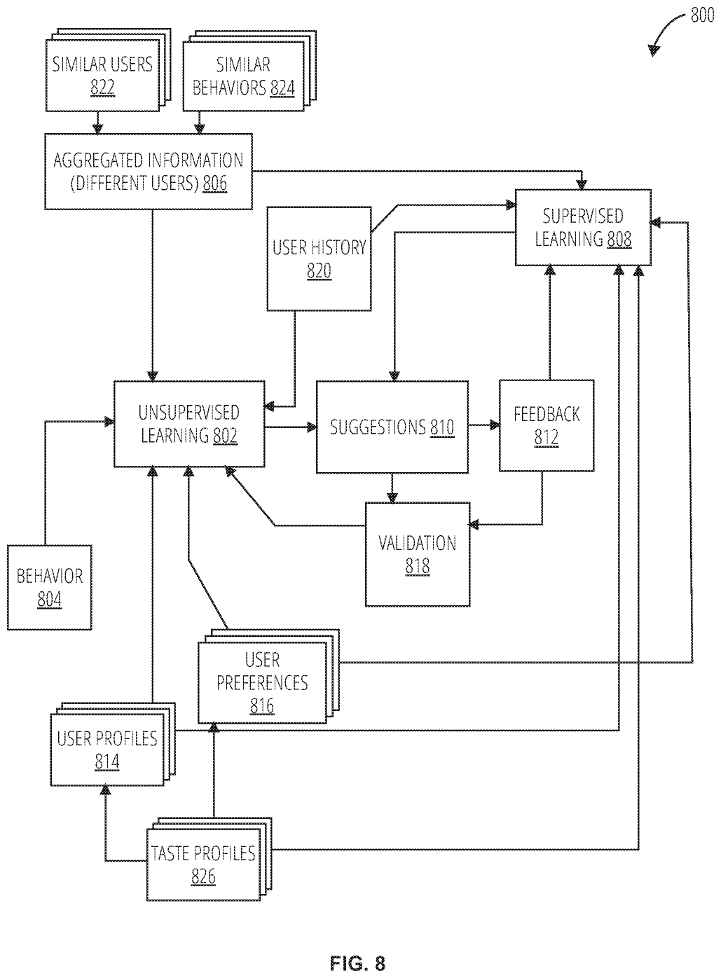

Referencing FIG. 8, a system 800 illustrates a behavior learning process in which aggregated information (different users) 806, user data, may be collected from similar users 822 with similar behaviors 824 and runs an unsupervised learning process 802 and a supervised learning process 808. The unsupervised learning process 802 incorporates behavior 804, user profiles 814, user preferences 816, and user history 820 to generate suggestions 810. The suggestions 810 may be provided with feedback 812, which serves as validation 818 for the unsupervised learning process 802. The supervised learning process 808 may incorporate the user preferences 816, user profiles 814, and user history 820 to generate suggestions 810. The suggestions 810 may be provided with feedback 812, which helps improve the supervised learning process 808. The user profiles 814 and the user preferences 816 may also include taste profiles 826 utilized by either the unsupervised learning process 802 or the supervised learning process 808 for generating suggestions 810.

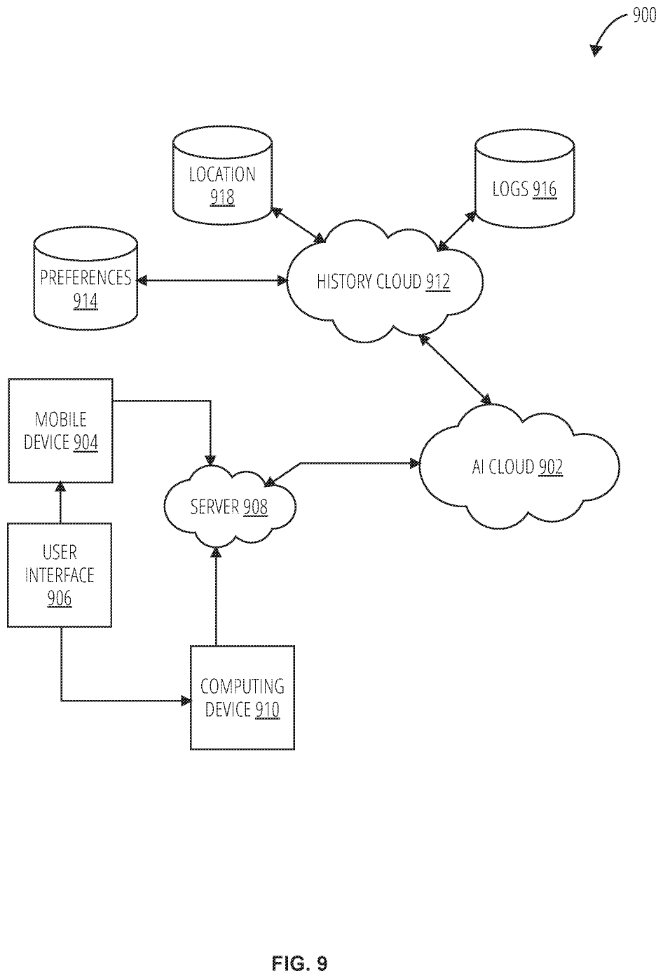

Referencing FIG. 9, a system 900 involves a history cloud 912 that bidirectionally communicates with system logs 916, location 918, and preferences 914 storage. A server 908 may receive user interactions via a user interface 906 by way of a mobile device 904 or a computing device 910. The server 908 may communicate the user information to an artificial intelligence (AI) cloud 1102, which may correlate the user interactions received from the server to information stored on the history cloud 912.

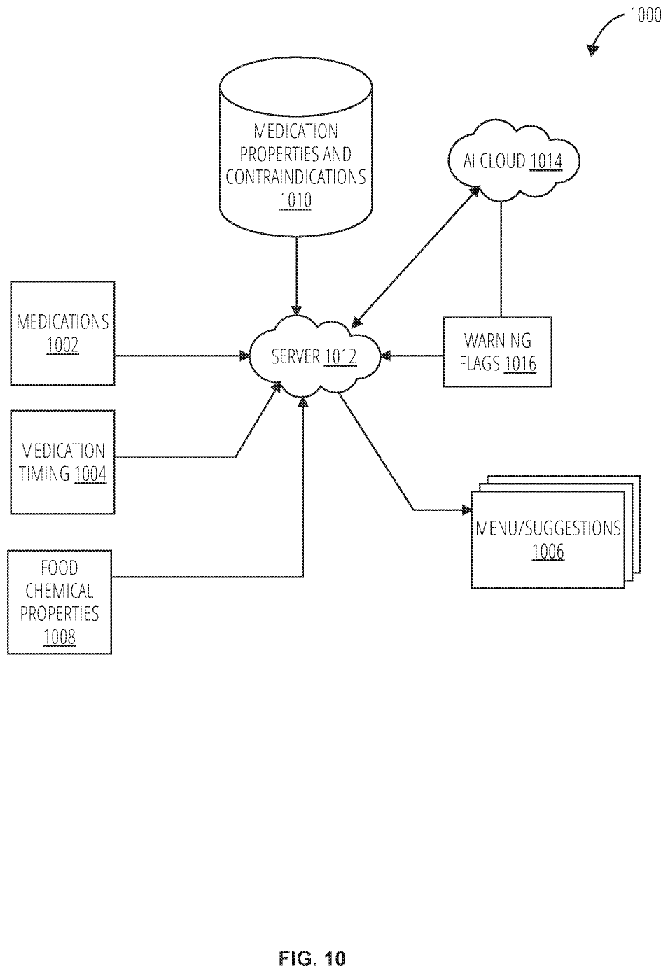

Referencing FIG. 10, a system 1000 illustrates how a server 1012 receives information regarding medications 1002, medication timing 1004, and food chemical properties 1008, and references those inputs with a database containing medication properties and contraindications 1010. The server 1012 may also provide the information to an AI cloud 1014 which may help in the generation of a menu or food suggestions 1006.

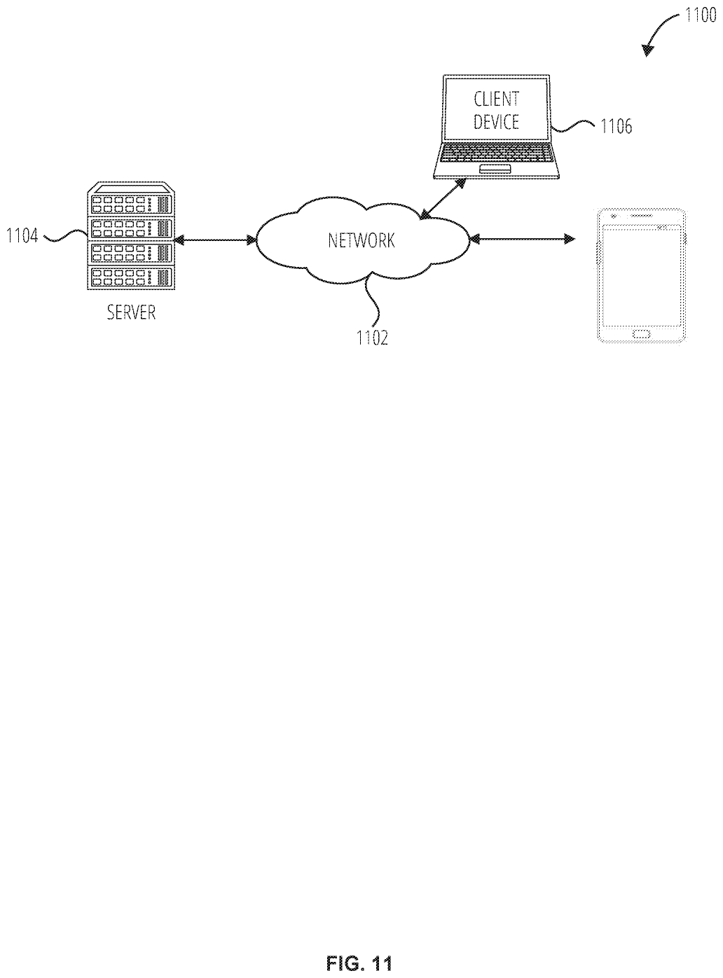

FIG. 11 illustrates a system 100 in which a server 1104 and a client device 1106 are connected to a network 1102.

In various embodiments, the network 1102 may include the Internet, a local area network (“LAN”), a wide area network (“WAN”), and/or other data network. In addition to traditional data-networking protocols, in some embodiments, data may be communicated according to protocols and/or standards including near field communication (“NFC”), Bluetooth, power-line communication (“PLC”), and the like. In some embodiments, the network 1102 may also include a voice network that conveys not only voice communications, but also non-voice data such as Short Message Service (“SMS”) messages, as well as data communicated via various cellular data communication protocols, and the like.

In various embodiments, the client device 1106 may include desktop PCs, mobile phones, laptops, tablets, wearable computers, or other computing devices that are capable of connecting to the network 1102 and communicating with the server 1104, such as described herein.

In various embodiments, additional infrastructure (e.g., short message service centers, cell sites, routers, gateways, firewalls, and the like), as well as additional devices may be present. Further, in some embodiments, the functions described as being provided by some or all of the server 1104 and the client device 1106 may be implemented via various combinations of physical and/or logical devices. However, it is not necessary to show such infrastructure and implementation details in FIG. 11 in order to describe an illustrative embodiment.

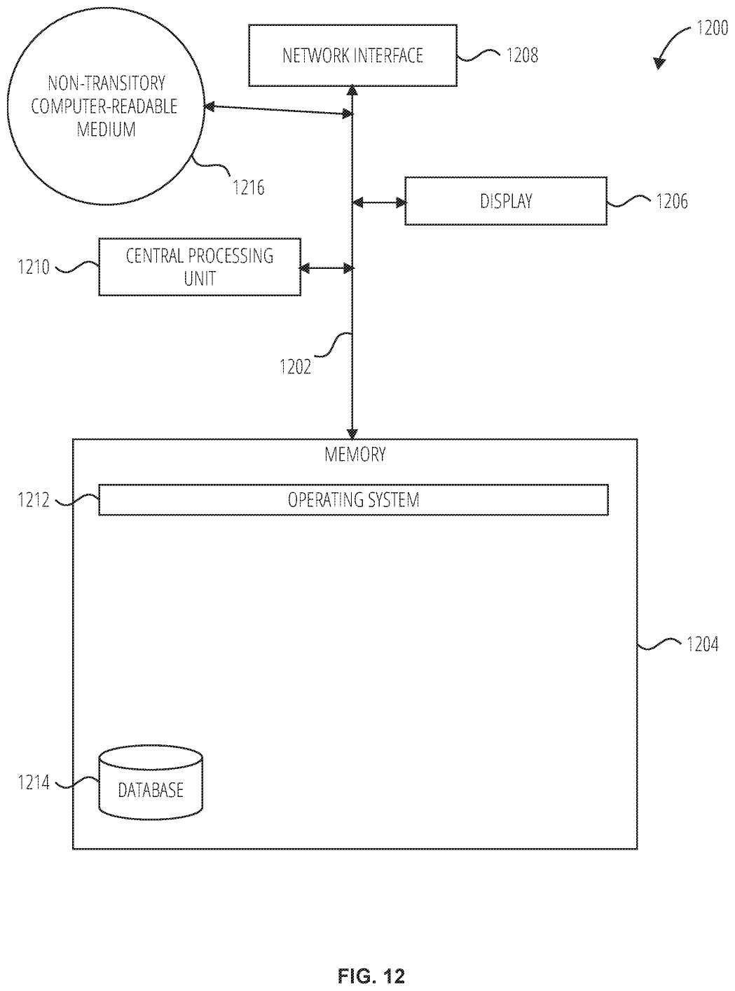

FIG. 12 illustrates several components of an exemplary system 1200 in accordance with one embodiment. In various embodiments, system 1200 may include a desktop PC, server, workstation, mobile phone, laptop, tablet, set-top box, appliance, or other computing device that is capable of performing operations such as those described herein. In some embodiments, system 1200 may include many more components than those shown in FIG. 12. However, it is not necessary that all of these generally conventional components be shown in order to disclose an illustrative embodiment. Collectively, the various tangible components or a subset of the tangible components may be referred to herein as “logic” configured or adapted in a particular way, for example as logic configured or adapted with particular software or firmware.

In various embodiments, system 1200 may comprise one or more physical and/or logical devices that collectively provide the functionalities described herein. In some embodiments, system 1200 may comprise one or more replicated and/or distributed physical or logical devices.

In some embodiments, system 1200 may comprise one or more computing resources provisioned from a “cloud computing” provider, for example, Amazon Elastic Compute Cloud (“Amazon EC2”), provided by Amazon.com, Inc. of Seattle, Wash.; Sun Cloud Compute Utility, provided by Sun Microsystems, Inc. of Santa Clara, Calif.; Windows Azure, provided by Microsoft Corporation of Redmond, Wash., and the like.

System 1200 includes a bus 1202 interconnecting several components including a network interface 1208, a display 1206, a central processing unit 1210, and a memory 1204.

Memory 1204 generally comprises a random access memory (“RAM”) and permanent non-transitory mass storage device, such as a hard disk drive or solid-state drive. Memory 1204 stores an operating system 1212.

These and other software components may be loaded into memory 1204 of system 1200 using a drive mechanism (not shown) associated with a non-transitory computer-readable medium 1216, such as a DVD/CD-ROM drive, memory card, network download, or the like.

Memory 1204 also includes database 1214. In some embodiments, system 1200 may communicate with database 1214 via network interface 1208, a storage area network (“SAN”), a high-speed serial bus, and/or via the other suitable communication technology.

In some embodiments, database 1214 may comprise one or more storage resources provisioned from a “cloud storage” provider, for example, Amazon Simple Storage Service (“Amazon S3”), provided by Amazon.com, Inc. of Seattle, Wash., Google Cloud Storage, provided by Google, Inc. of Mountain View, Calif., and the like.

Terms used herein should be accorded their ordinary meaning in the relevant arts, or the meaning indicated by their use in context, but if an express definition is provided, that meaning controls.

“Circuitry” refers to electrical circuitry having at least one discrete electrical circuit, electrical circuitry having at least one integrated circuit, electrical circuitry having at least one application specific integrated circuit, circuitry forming a general purpose computing device configured by a computer program (e.g., a general purpose computer configured by a computer program which at least partially carries out processes or devices described herein, or a microprocessor configured by a computer program which at least partially carries out processes or devices described herein), circuitry forming a memory device (e.g., forms of random access memory), or circuitry forming a communications device (e.g., a modem, communications switch, or optical-electrical equipment).

“Firmware” refers to software logic embodied as processor-executable instructions stored in read-only memories or media.

“Hardware” refers to logic embodied as analog or digital circuitry.

“Logic” refers to machine memory circuits, non transitory machine readable media, and/or circuitry which by way of its material and/or material-energy configuration comprises control and/or procedural signals, and/or settings and values (such as resistance, impedance, capacitance, inductance, current/voltage ratings, etc.), that may be applied to influence the operation of a device. Magnetic media, electronic circuits, electrical and optical memory (both volatile and nonvolatile), and firmware are examples of logic. Logic specifically excludes pure signals or software per se (however does not exclude machine memories comprising software and thereby forming configurations of matter).

“Software” refers to logic implemented as processor-executable instructions in a machine memory (e.g. read/write volatile or nonvolatile memory or media).

Herein, references to “one embodiment” or “an embodiment” do not necessarily refer to the same embodiment, although they may. Unless the context clearly requires otherwise, throughout the description and the claims, the words “comprise,” “comprising,” and the like are to be construed in an inclusive sense as opposed to an exclusive or exhaustive sense; that is to say, in the sense of “including, but not limited to.” Words using the singular or plural number also include the plural or singular number respectively, unless expressly limited to a single one or multiple ones. Additionally, the words “herein,” “above,” “below” and words of similar import, when used in this application, refer to this application as a whole and not to any particular portions of this application. When the claims use the word “or” in reference to a list of two or more items, that word covers all of the following interpretations of the word: any of the items in the list, all of the items in the list and any combination of the items in the list, unless expressly limited to one or the other. Any terms not expressly defined herein have their conventional meaning as commonly understood by those having skill in the relevant art(s).

Various logic functional operations described herein may be implemented in logic that is referred to using a noun or noun phrase reflecting said operation or function. For example, an association operation may be carried out by an “associator” or “correlator”. Likewise, switching may be carried out by a “switch”, selection by a “selector”, and so on.

What is claimed is:

1. A method of operating a meal plan optimization algorithm, the method comprising:
retrieving at least one user profile from a profile database;
configuring a menu generation algorithm with user preferences from the at least one user profile;
configuring a selector to retrieve food items from at least one proximal food database for the menu generation algorithm from the user preferences;
operating the menu generation algorithm, configured by the user preferences, to:
set preferred food items for meal components of individual meals from the food items in the at least one proximal food database through operation of a food preference discriminator;
set a non-user specified food item for a meal component of the individual meals without a preferred food item and additional food items through operation of the food preference discriminator;
update a meal total for the individual meals with the non-user specified food item;
operate a meal target evaluator iteratively, through operation of an iterator, to adjust and substitute meal components; and
generate a meal day plan from the individual meals;
adjusting a portion size for the individual meals in the meal day plan through operation of a kcal target evaluator, configured by the user preferences; and
generating a food menu for at least one day, displayable through a user interface associated with the at least one user profile, from meal day plans through operation of a constructor.

2. The method of claim 1 further comprising adjusting the portion size of the individual meals in the meal day plan for each user profile through operation of the kcal target evaluator configured by the user preferences from each user profile.

3. The method of claim 1, wherein the user preferences comprise food dislikes, food likes, food allergies or restrictions, meal preferences, nutrient targets, weight or other personal health objectives, preferred grocers or food distributors, preferred brands or private labels, financial budget, and kcal target from the at least one user profile.

4. The method of claim 1, further comprising:
receiving an indication of a consumed food item not included in the food menu; and
operating the meal target evaluator to adjust or substitute components to modify the meal day plan through operation of the kcal target evaluator.

5. The method of claim 4, wherein adjust or substitute components comprises adjusting the portion size of a subsequent meal.

6. The method of claim 1, further comprising determining nutritional values for the individual meals.

7. The method of claim 6, further comprising determining a nutritional target and identifying a nutritional gap between the nutritional target and the nutritional values for the individual meals.

8. The method of claim 7, further comprising generating additional individual meals in response to identifying the nutritional gap, the additional individual meals generated to reduce the nutritional gap.

9. The method of claim 1, further comprising generating a shopping list for the individual meals in the meal day plan.

10. The method of claim 1, where retrieving at least one user profile from a profile database comprises retrieving two or more user profiles from the profile database and generate the food menu comprises generating a food menu for the two or more user profiles.

11. The method of claim 1, further comprising determining a portion size for the individual meals, based at least in part on a number of user profiles retrieved from the profile database.

12. A method of creating a meal plan, comprising:
retrieving one or more user profiles comprising user preferences;
retrieving data associated with food items from a proximal food database;
generate one or more individual meals based at least in part on the user preferences and the food items;
generate a meal total for each of the individual meals, the meal total comprising nutritional information associated with the meal;
comparing the meal total with a meal target;
iteratively modify the individual meals until the meal total exceeds the meal target;
generate a food menu for a day, the food menu comprising individual meals and displayable through a user interface associated with a user.

13. The method of claim 12, further comprising receiving feedback through the user interface and modifying the food menu in response to the feedback.

14. The method of claim 12, further comprising retrieving a food inventory of a food distributor.

15. The method of claim 14, further comprising generating a shopping list based on the food menu and food inventory of the food distributor.

16. The method of claim 14, further comprising determining that the food inventory does not include one or more food items within the food menu.

17. The method of claim 16, further comprising iteratively modifying the individual meals until the food inventory includes all the food items within the individual meals.

18. A method, comprising:
receiving a user profile comprising user food preferences;
receiving food items from a proximal food database;
operating a menu generation algorithm to generate an individual meal in response to receiving the food items and receiving the user profile;
determine a kcal target associated with the user profile;
compare the individual meal to the kcal target;
adjust a portion size of the individual meal; and
generate a second individual meal based on the portion size.

19. The method of claim 18, further comprising iterating the step of operating the menu generation algorithm to create a meal plan comprising a plurality of individual meals.

20. The method of claim 19, further comprising:
logging the plurality of individual meals;
generating a meal score of the plurality of individual meals; and
generating additional meals in response to the meal score of the plurality of individual meals.
